(19) United States
(12) Patent Application Publication
(10) Pub. No.: US 2022/0287584 A1
(43) Pub. Date: Sep. 15, 2022

(54) Methods for Diagnosis and Treatment of Deep Tissue Injury Using Sub-Epidermal Moisture Measurements

(71) Applicant: BRUIN BIOMETRICS, LLC, Los Angeles, CA (US)

(72) Inventors: Martin F. BURNS, Los Angeles, CA (US); Vignesh Mani IYER, Torrance, CA (US)

(73) Assignee: BRUIN BIOMETRICS, LLC, Los Angeles, CA (US)

(21) Appl. No.: 17/689,580

(22) Filed: Mar. 8, 2022

Related U.S. Application Data
(60) Provisional application No. 63/158,713, filed on Mar. 9, 2021.
(60) Provisional application No. 63/316,218, filed on Mar. 3, 2022.

(51) Int. Cl. A61B 5/0537; A61B 5/00; G16H 50/20; G06N 3/04; G06N 3/08

(52) U.S. Cl. CPC A61B 5/0537; A61B 5/445; A61B 5/7275; G16H 50/20; G06N 3/0472; G06N 3/08

(57) ABSTRACT

The present disclosure provides methods, apparatuses and computer readable media for measuring sub-epidermal moisture in patients to determine deep tissue injury for clinical intervention. The present disclosure also provides methods for detecting and predicting deep tissue injury. The present disclosure further provides methods for determining appropriate clinical intervention including preventative measures and treatments of deep tissue injury.

### CROSS REFERENCE TO RELATED APPLICATIONS

This application claims priority to U.S. Provisional Application No. 63/158,713 filed Mar. 9, 2021, and U.S. Provisional Application No. 63/316,218 filed Mar. 3, 2022, the entirety of each of which is herein incorporated by reference.

### FIELD OF INVENTION

The present disclosure provides systems, apparatuses, and computer readable media for measuring sub-epidermal moisture in patients to determine damaged tissue for clinical intervention. The present disclosure also provides methods for determining damaged tissue. The present disclosure further provides methods for determining appropriate clinical intervention including preventative measures and treatments for deep tissue injuries.

### BACKGROUND

The skin is the largest organ in the human body. It is readily exposed to different kinds of damages and injuries. When the skin and its surrounding tissues are unable to redistribute external pressure and mechanical forces, pressure ulcers (also known as pressure injuries or bedsores) may be formed. Pressure ulcers pose a significant health and economic concern internationally, across both acute and long-term care settings. Pressure ulcers impact approximately 2.5 million people a year in the United States and an equivalent number in the European Union. In long-term and critical care settings, up to 25% of elderly and immobile patients develop pressure ulcers. Approximately 60,000 U.S. patients die per year due to infection and other complications from pressure ulcers.

Most pressure ulcers occur over bony prominences, where there is less tissue for compression and the pressure gradient within the vascular network is altered. Pressure ulcers are categorized in one of four stages, ranging from the earliest stage currently recognized, in which the skin remains intact but may appear red over a bony prominence (Stage 1), to the last stage, in which tissue is broken and bone, tendon or muscle is exposed (Stage 4). In addition to the four stages of pressure ulcers, there is also a unique form of ulcers known as deep tissue injury (DTI) or deep tissue pressure injury (DTPI). See, e.g., “Deep Tissue Injury,” National Pressure Ulcer Advisory Panel (NPUAP) White Paper, available at npiap.com (2004). DTI is a pressure-related injury to subcutaneous tissues under intact skin. These injuries have the appearance of a deep bruise, and are initiated in the muscle layer next to a bony prominence, developing outwards towards the epidermis layer. Due to the etiology of DTI, it is often difficult to accurately classify DTI into any one of the 4 stages above. DTI may also develop in a relative short period of time and often deteriorates rapidly into Stage 3 and Stage 4 ulcers.

Currently, DTI is diagnosed through visual examination for purple or maroon localized area of discolored intact skin or blood-filled blister due to damage of the underlying soft tissue. Accordingly, DTI may be difficult to detect in individuals with dark skin tones. Moreover, visual examination of DTI may also lead to misclassification under Stage 1 or Stage 2 ulcers, which does not represent the true and dangerous potential of the injury.

To treat pressure ulcers in a timely and effective manner, clinicians need to be able to identify, with precision, the presence of a DTI, and distinguish it from the early stages of ulcers. However, the current standard to detect pressure ulcers is by visual inspection, which is subjective, unreliable, untimely, and lacks specificity.

### SUMMARY OF THE INVENTION

The present disclosure provides for, and includes, systems, apparatuses, and methods for detecting deep tissue injury (DTI). In an aspect, the present disclosure provides 1) a method for detecting deep tissue injury (DTI) before it is visible on a patient's skin, comprising: a) obtaining a set of SEM delta values at a location on the patient's skin at a predetermined frequency; b) applying to each of the SEM delta values of the obtained set a predetermined weight; c) calculating a first average SEM delta value of the N least-recent weighted SEM delta values; d) calculating a second average SEM delta value of the M most-recent weighted SEM delta values; e) comparing the difference between the first average and the second average SEM delta value with a predetermined threshold value; and f) determining that there is DTI at the location on the patient's skin when the difference is greater than the predetermined threshold value. In an aspect, the set of obtained SEM delta values comprises at least five SEM delta values each taken one day apart. In an aspect, the predetermined weights are in the range of 0 to 2. In an aspect, the predetermined weights monotonically increase with time. In an aspect, N>M. In an aspect, N is 4 and M is 2. In an aspect, the predetermined threshold value is a real number in the range of 0 to 1. In an aspect, the most recent SEM delta value is obtained by linear extrapolation of the K most-recent SEM delta values. In an aspect, K is 3. In an aspect, the predetermined threshold is 0.7. In an aspect, the location on the patient's skin is selected from the group consisting of the back of the head, the sacrum, the shoulder, the elbow, the lower back, the tailbone, the buttock, the hip, the inner knee, the heel, and a combination thereof.

The present disclosure also provides for a computer-implemented method for training a neural network for detection of deep tissue injury (DTI) before the injury is visible on a patient's skin, comprising: a) for each patient in a first plurality of patients who have been diagnosed with a DTI, obtaining a first set of sub-epidermal moisture (SEM) delta values at a location on the patient's skin at a predetermined frequency before and up to the formation of the DTI; b) for each patient in a second plurality of patients who have not been diagnosed with a DTI, obtaining a second set of SEM delta values at the same location on the patient's skin at the predetermined frequency; c) applying a set of weights to each of the SEM delta values of the first and second set of obtained SEM delta values; d) creating a training set comprising the first set of weighted SEM delta values and the second set of weighted SEM delta values of all patients in the first and second plurality of patients; and e) training the neural network using the training set. In an aspect, the trained neural network outputs a set of optimized weights, comprising an optimized weight for each timepoint before the formation of a DTI, and where the optimized weights monotonically increase with time. In an aspect, the method further comprises the steps of: a) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; b) creating a training set comprising a first set of difference values in the first plurality of patients and a second set of difference values in the second plurality of patients; and c) training the neural network using the training set. In an aspect, the predetermined frequency is once a day. In an aspect, the first and second set of SEM delta values comprises at least six SEM delta values each taken one day apart. In an aspect, N+M =6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof.

The present disclosure also provides a computer-implemented method for training a neural network for detection of deep tissue injury (DTI) before the injury is visible on a patient's skin, comprising: a) for each patient in a first plurality of patients who experience a DTI, obtaining a first set of sub-epidermal moisture (SEM) delta values at a location on the patient's skin at a predetermined frequency before and up to the formation of the DTI; b) for each patient in a second plurality of patients who do not experience a DTI, obtaining a second set of SEM delta values at the same location on the patient's skin at the predetermined frequency; c) applying a set of weights to each of the SEM delta values of the first and second set of obtained SEM delta values; d) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; e) creating a training set comprising a first set of difference values in the first plurality of patients and a second set of difference values in the second plurality of patients; and f) training the neural network using the training set. In an aspect, the trained neural network outputs a) a set of optimized weights, comprising an optimized weight for each timepoint before the formation of a DTI, where the optimized weights monotonically increase with time; b) an optimized threshold; c) the value of N; and d) the value of M. In an aspect, the predetermined frequency is once a day. In an aspect, the first and second set of SEM delta values comprises at least six SEM delta values each taken one day apart. In an aspect, N+M=6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof. In an aspect, the set of optimized weights and the optimized threshold are used to predict the occurrence of deep tissue injury.

The present disclosure provides for, and includes, a non-transitory computer readable medium for identifying DTI tissue. The non-transitory computer readable medium may comprise instructions stored thereon, that when executed on a processor, may perform the steps of: a) obtaining a set of SEM delta values at a location on the patient's skin at a predetermined frequency; b) applying to each of the SEM delta values of the obtained set a predetermined weight; c) calculating a first average SEM delta value of the N least-recent weighted SEM delta values; d) calculating a second average SEM delta value of the M most-recent weighted SEM delta values; e) comparing the difference between the first average and the second average SEM delta value with a predetermined threshold value; and f) determining that there is DTI at the location on the patient's skin when the difference is greater than the predetermined threshold value. In an aspect, the set of obtained SEM delta values comprises at least five SEM delta values each taken one day apart. In an aspect, the predetermined weights are in the range of 0 to 2. In an aspect, the predetermined weights monotonically increase with time. In an aspect, N>M. In an aspect, N is 4 and M is 2. In an aspect, the predetermined threshold value is a real number in the range of 0 to 1. In an aspect, the most recent SEM delta value is obtained by linear extrapolation of the K most-recent SEM delta values. In an aspect, K is 3. In an aspect, the predetermined threshold is 0.7. In an aspect, the location on the patient's skin is selected from the group consisting of the back of the head, the sacrum, the shoulder, the elbow, the lower back, the tailbone, the buttock, the hip, the inner knee, the heel, and a combination thereof.

The present disclosure provides for, and includes, a non-transitory computer readable medium for identifying DTI tissue. The non-transitory computer readable medium may comprise instructions stored thereon, that when executed on a processor, may perform the steps of: a) for each patient in a first plurality of patients who have been diagnosed with a DTI, obtaining a first set of sub-epidermal moisture (SEM) delta values at a location on the patient's skin at a predetermined frequency before and up to the formation of the DTI; b) for each patient in a second plurality of patients who have not been diagnosed with a DTI, obtaining a second set of SEM delta values at the same location on the patient's skin at the predetermined frequency; c) applying a set of weights to each of the SEM delta values of the first and second set of obtained SEM delta values; d) creating a training set comprising the first set of weighted SEM delta values and the second set of weighted SEM delta values of all patients in the first and second plurality of patients; and e) training the neural network using the training set. In an aspect, the trained neural network outputs a set of optimized weights, comprising an optimized weight for each timepoint before the formation of a DTI, and where the optimized weights monotonically increase with time. In an aspect, the method further comprises the steps of: a) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; b) creating a training set comprising a first set of difference values in the first plurality of patients and a second set of difference values in the second plurality of patients; and c) training the neural network using the training set. In an aspect, the predetermined frequency is once a day. In an aspect, the first and second set of SEM delta values comprises at least six SEM delta values each taken one day apart. In an aspect, N+M=6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof

The present disclosure provides for, and includes, a non-transitory computer readable medium for identifying DTI tissue. The non-transitory computer readable medium may comprise instructions stored thereon, that when executed on a processor, may perform the steps of: a) for each patient in a first plurality of patients who experience a DTI, obtaining a first set of sub-epidermal moisture (SEM) delta values at a location on the patient's skin at a predetermined frequency before and up to the formation of the DTI; b) for each patient in a second plurality of patients who do not experience a DTI, obtaining a second set of SEM delta values at the same location on the patient's skin at the predetermined frequency; c) applying a set of weights to each of the SEM delta values of the first and second set of obtained SEM delta values; d) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; e) creating a training set comprising a first set of difference values in the first plurality of patients and a second set of difference values in the second plurality of patients; and f) training the neural network using the training set. In an aspect, the trained neural network outputs a) a set of optimized weights, comprising an optimized weight for each timepoint before the formation of a DTI, where the optimized weights monotonically increase with time; b) an optimized threshold; c) the value of N; and d) the value of M. In an aspect, the predetermined frequency is once a day. In an aspect, the first and second set of SEM delta values comprises at least six SEM delta values each taken one day apart. In an aspect, N+M=6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof. In an aspect, the set of optimized weights and the optimized threshold are used to predict the occurrence of deep tissue injury.

The present disclosure provides for, and includes, a computer-implemented method for predicting deep tissue injury (DTI), the method comprising: receiving, via an input device, a plurality of sub-epidermal moisture (SEM) delta values associated with a patient; automatically inputting, via a processor, the plurality of SEM delta values into a trained model to receive a probability value, where the model is configured to predict a probability value corresponding to a future occurrence of the patient developing DTI, where the model is trained based on a set of training data comprising SEM delta data from a set of patients; and outputting, via an output device, a prediction of the future occurrence of the patient developing DTI based on the probability value. In an aspect, training the model comprises the steps of: receiving, via an input device, a set of training data comprising: 1) a plurality of sub-epidermal moisture (SEM) delta values associated with a set of patients, and 2) a threshold value, where each patient in the set of patients has a known deep tissue injury (DTI) status, where the threshold value is a number between 0 and 1; automatically inputting, via a processor, the training data into an optimization algorithm to receive a plurality of optimal weight values; and automatically updating, via a processor, the model with the plurality of optimal weight values. In an aspect, the optimization algorithm is configured to: generate a plurality of increasing random numbers between 0 and 2 as a plurality of weight values; input the training data and the plurality of weight values into the model to receive a set of predicted deep tissue injury (DTI) statuses associated with the set of patients; compare the predicted DTI statuses with the known DTI statuses associated with the set of patients; calculate a true positive rate (TPR) and a false positive rate (FPR) based on the comparison, where the TPR is calculated as percentage of patients in the set of patients whose predicted DTI status matches their known DTI status, and the FPR is calculated as percentage of patients in the set of patients whose predicted DTI status does not match their known DTI status; repeat steps a) to d) for a predetermined number of times as the number of iterations; identify optimal TPR and FPR from all calculated TPRs and FPRs from the iterations; and output the plurality of optimal weight values associated with the identified optimal TPR and FPR. In an aspect, the plurality of SEM delta values comprises the SEM delta readings from a predetermined number of days before the day of predicting deep tissue injury (DTI). In an aspect, identifying optimal TPR and FPR comprises minimizing the objective function of 1-TPR+FPR and satisfying the constraint of TPR»FPR. In an aspect, the input device is a SEM scanner. In an aspect, the SEM scanner is connected to a computer by cable or by wireless technology. In an aspect, the DTI occurs at a location selected from the group consisting of the back of the head, the sacrum, the shoulder, the elbow, the lower back, the tailbone, the buttock, the hip, the inner knee, the heel, and a combination thereof.

The present disclosure provides for, and includes, a method for assessing risk of deep tissue injury (DTI) of a patient, the method comprising: obtaining a plurality of sub-epidermal moisture (SEM) delta values associated with a patient; inputting the plurality of SEM delta values into a trained model to receive a probability value, where the model is configured to predict a probability value corresponding to future occurrence of the patient developing DTI, where the model is trained based on a set of training data comprising SEM delta data of a set of patients; outputting a prediction of future DTI occurrence likelihood of the patient based on the probability value; and assessing risk of the patient developing DTI based on the predicted future DTI occurrence likelihood. In an aspect, the predicted future occurrence is categorized as no DTI, low likelihood of DTI, high likelihood of DTI, or suspected DTI. In an aspect, the method further comprises selecting an intervention for the patient based on the assessed risk of DTI. In an aspect, the intervention comprises at least one of: reducing pressure, repositioning the patient, changing the patient's support surface, providing a low-friction padded mattress, providing a silicon pad, providing a heel boot, cleaning and dressing wounds, removing damaged tissue, applying a topical cream, applying a barrier cream, applying neuro-muscular stimulation, drug administration, and surgery. In an aspect, the DTI occurs at a location selected from the group consisting of the back of the head, the sacrum, the shoulder, the elbow, the lower back, the tailbone, the buttock, the hip, the inner knee, the heel, and a combination thereof. In an aspect, the method further comprises generating or updating a report from performing the method. In an aspect, the method further comprises transmitting the report to the patient or a clinician. In an aspect, the method further comprises storing the report on a non-transitory computer readable storage medium. In an aspect, the method further comprises displaying the report on a computer display.

The present disclosure provides for, and includes, a non-transitory computer-readable storage medium comprising one or more programs for execution by one or more processors of a device, the one or more programs comprising instructions which, when executed by the one or more processors, cause the device to perform the method of any one of the preceding embodiments. In an aspect, the non-transitory computer-readable storage medium further comprises a report generated from performing the method of any one of the preceding embodiments.

The present disclosure provides for, and includes, an electronic device, comprising: one or more processors; a memory; and one or more programs, where the one or more programs comprises instructions which, when executed by the one or more processors, cause the device to perform the method of any one of the preceding embodiments. In an aspect, the electronic device further comprises one or more displays to present a report generated from performing the method of any one of the preceding embodiments.

### BRIEF DESCRIPTION OF THE FIGURES

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, make apparent to those skilled in the art how aspects of the disclosure may be practiced.

FIG. 1A: An example apparatus according to the present disclosure, comprising one coaxial electrode.

FIG. 1B: An illustration of an example method using an example apparatus according to the present disclosure to measure SEM delta values, in order to detect potential DTIs.

FIG. 2A: Sample SEM delta values taken from patients who were diagnosed with DTIs (left) and patients who were not diagnosed with DTIs (right).

FIG. 2B: An illustration of an example method for detecting DTIs according to the present disclosure.

FIG. 3: Sample SEM delta values taken from patients.

FIG. 4: Example ways of dividing data for processing.

FIG. 5A: A flowchart illustrating an example method for predicting DTI according to the present disclosure.

FIG. 5B: A flowchart illustrating an example method for optimizing weights according to the present disclosure.

FIG. 6: Sample optimized weights and threshold according to the present disclosure.

FIG. 7: Example method for predicting formation of DTIs according to the present disclosure.

FIG. 8: Results of example prediction and detection methods according to the present disclosure.

FIG. 9: An example system in accordance with an aspect of the present disclosure.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations and variations thereof

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein comprise one or more steps or actions for achieving the described method. The method steps and/or actions may be interchanged with one another without departing from the scope of the present invention. In other words, unless a specific order of steps or actions is required for proper operation of the embodiment, the order and/or use of specific steps and/or actions may be modified without departing from the scope of the present invention.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM delta value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “exemplary” is used to mean serving as an example, instance, or illustration. Any aspect or aspect described as “exemplary” is not necessarily to be construed as preferred or advantageous over other aspects or aspects, nor is it meant to preclude equivalent structures and techniques known to those of ordinary skill in the art. Rather, use of the word exemplary is intended to present concepts in a concrete fashion, and the disclosed subject matter is not limited by such examples.

As used herein, the term “sub-epidermal moisture” or “SEM” refers to the amount of moisture in skin tissue under the epidermis. SEM may include intracellular and extracellular fluid. Without being bound by theory, when skin tissue is damaged, inflammation at the site of injury can cause blood vessels to dilate and increase blood flow into the skin tissue. There can also be an increase in blood vessel permeability, allowing fluid, proteins, and white blood cells to migrate from the circulation to the site of skin tissue damage. The flood of fluids, cells, and other substances to the injured site produces swelling and redness, and increases the amount of SEM in the damaged skin tissue. Processes like apoptosis and necrosis may also increase the amount of fluid in the damaged region. SEM may be measured, for example, by measurement of biocapacitance. In some aspects, the methods described herein comprise a step of obtaining an SEM value in a tissue. In some aspects, the methods described herein comprise a step of obtaining an SEM value in the skin. In some aspects, the methods described herein comprise a step of obtaining an SEM value in the sub-epidermal layer of the skin. In some aspects, the methods described herein comprise a step of obtaining an SEM delta value in a tissue. In some aspects, the methods described herein comprise a step of obtaining an SEM delta value in the skin. In some aspects, the methods described herein comprise a step of obtaining a set of SEM values in a tissue. In some aspects, the methods described herein comprise a step of obtaining a set of SEM values in the skin. In some aspects, the methods described herein comprise a step of obtaining a set of SEM delta values in a tissue. In some aspects, the methods described herein comprise a step of obtaining a set of SEM delta values in the skin. In some aspects, a set of SEM delta values comprises a plurality of SEM delta values taken at different times. In some aspects, a set of SEM delta values comprises a plurality of SEM delta values taken at the same time.

As used herein, “SEM delta value” or “SEM-A value” refers to a calculated difference between two values derived from SEM measurements obtained at the same tissue location or at approximately the same time regardless of tissue location.

In an aspect, each of the two values is a SEM measurement value obtained at approximately the same time as the other. In an aspect, each of the two values is an average value determined from a subset of a plurality of SEM measurement values obtained at approximately the same time. In an aspect, the two values are an SEM measurement value and an average value determined from a plurality of SEM measurement values obtained at approximately the same time. In an aspect, the two values are a maximum SEM measurement value and a SEM measurement value obtained at approximately the same time. In an aspect, the two values are a maximum SEM measurement value and an average value determined from a plurality of SEM measurement values obtained at approximately the same time. In an aspect, the two values are a maximum SEM measurement value and a minimum SEM measurement value determined from a plurality of SEM measurement values obtained at approximately the same time.

In an aspect, each of the two values is a SEM measurement value obtained at the same tissue location. In an aspect, each of the two values is an average SEM value determined from a subset of a plurality of SEM measurement values obtained at the same tissue location. In an aspect, the two values are an SEM measurement value and an average value determined from a plurality of SEM measurement values obtained at the same tissue location. In an aspect, the two values are a maximum SEM measurement value and a SEM measurement value obtained at the same tissue location. In an aspect, the two values are a maximum SEM measurement value and an average value determined from a plurality of SEM measurement values obtained at the same tissue location. In an aspect, the two values are a maximum SEM measurement value and a minimum SEM measurement value determined from a plurality of SEM measurement values obtained at the same tissue location. In an aspect, two SEM measurements obtained at the same tissue location are measurements taken at spatially distinct locations on the tissue. In an aspect, two SEM measurements obtained at the same tissue location are measurements taken at overlapping locations on the tissue. In an aspect, the tissue location is centered on an anatomical site, including but not limited to a sternum, sacrum, a heel, a scapula, an elbow, an ear, or other fleshy tissue. In an aspect, obtaining a plurality of SEM measurement at the same tissue location comprises taking measurements at and around an anatomical location. In an aspect, obtaining a plurality of SEM measurement at the same tissue location comprises taking measurements at and around an anatomical location based on a measurement map. Exemplary measurements maps may be found, for example, in U.S. patent application Ser. No. 17/591,139 or U.S. Pat. No. 9,763,596 B2, which are incorporated herein in their entireties.

In an aspect, an average SEM value is determined for a number of SEM values measured at approximately the same time at each spatial location surrounding an anatomical site. In an aspect, a SEM delta value is the difference between the maximum average SEM value and each of the average SEM values measured around the anatomical site at approximately the same time. In an aspect, a SEM delta value is the difference between the maximum average SEM value and the minimum average SEM value measured around the anatomical site at approximately the same time. In an aspect, “SEM delta value” may also be refer to a calculated difference between two values derived from measurements obtained at around the same tissue location at different times. In an aspect, two measurements are obtained at approximately the same time when they are taken no more than about 10 hours apart, no more than about 8 hours apart, no more than about 6 hours, no more than about 5 hours apart, no more than about 4 hours apart, no more than about 3 hours apart, no more than about 2 hours apart, or no more than about 1 hour apart.

As used herein, “tissue biocapacitance” refers to a biophysical marker for detecting initial tissue damage based on the increased level of fluids that build up in the interstitial space. Without being bound by theory, the greater the fluid content in a tissue, the higher the biocapacitance value becomes. In some aspects, the methods described herein comprise a step of measuring the biocapacitance in a tissue. In some aspects, the methods described herein comprise a step of measuring the biocapacitance of the skin. In some aspects, the biocapacitance measured with the methods described herein vary linearly with the SEM in the tissue. In some aspects, the biocapacitance measured with the methods described herein vary non-linearly with the SEM in the tissue.

As used herein, a “system” may be a collection of devices in wired or wireless communication with each other.

As used herein, “interrogate” refers to the use of radiofrequency energy to penetrate into a patient's skin.

As used herein, a “patient” may be a human or animal subject.

As used herein, a “weight” is a numerical coefficient assigned to an SEM delta value to express its relative importance in a distribution of SEM delta values. Without being bound by theory, a weight may adjust the contribution of SEM measurements on different days relative to the present day.

As used herein, a “neural network” is an artificial neural network comprising artificial neurons or nodes that uses a mathematical or computational model for information processing based on a connectionistic approach to computation. A neural network may perform supervised learning of a function that maps an input to an output based on example input-output pairs. The example input-output pairs may be given to the neural network as a “training set.”

As used herein, an “electrode sensor” is an electrode that detects an electrical property. In an aspect, an electrode sensor comprises one electrode. In an aspect, an electrode sensor comprises two electrodes. In an aspect, an electrode sensor comprises two electrodes placed in a coaxial configuration. In an aspect, an electrode sensor comprises two electrodes placed near each other.

As used herein, “prevention” or “preventing,” with respect to a condition or a disease, is an approach for reducing the risk of developing a condition or a disease before it manifests in a patient, or slowing and stopping the progression of the condition or disease once it has developed. Prevention approaches include, but are not limited to: identifying a disease at its earliest stage so that prompt and appropriate management can be initiated, protecting a tissue prone to a condition or a disease prior to its manifestation, reducing or minimizing the consequences of a disease, and a combination thereof. In an aspect, DTI is prevented when no DTI is formed. In an aspect, DTI is prevented when a DTI that has formed does not worsen.

As used herein, “treatment” or “treating,” with respect to a condition or a disease, is an approach for obtaining beneficial or desired results including preferably clinical results after a condition or a disease manifests in a patient. Beneficial or desired results with respect to a disease include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival, and a combination thereof. Likewise, for purposes of this disclosure, beneficial or desired results with respect to a condition include, but are not limited to, one or more of the following: improving a condition, curing a condition, lessening severity of a condition, delaying progression of a condition, alleviating one or more symptoms associated with a condition, increasing the quality of life of one suffering from a condition, prolonging survival, and a combination thereof

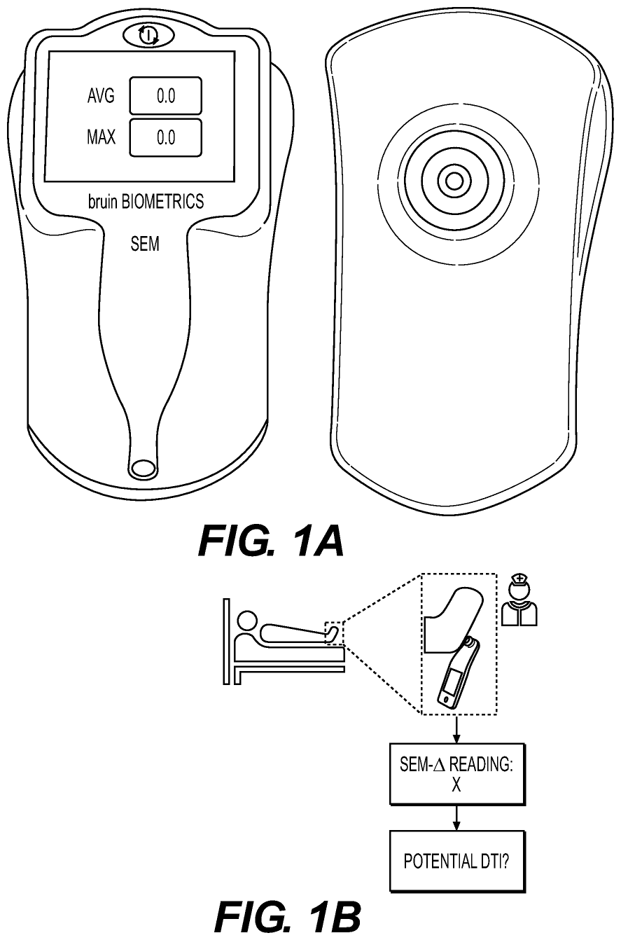

An exemplary apparatus according to the present disclosure is shown in FIGS. 1A and 1B. It will be understood that these are examples of an apparatus for measuring sub-epidermal moisture. In some embodiments, the apparatus according to the present disclosure may be a handheld device, a portable device, a wired device, a wireless device, or a device that is fitted to measure a part of a human patient. U.S. Pat. Nos. 9,220,455 B2 and 9,398,879 B2 to Sarrafzadeh et al., and U.S. Pat. No. 10,182,740 B2 to Tonar et al. are directed to different SEM scanning apparatuses. All of U.S. Pat Nos. 9,220,455 B2, 9,398,879B2 and 10,182,740B2 are incorporated herein by reference in their entireties.

### Methods of Detection

In an aspect, the present disclosure provides a method for detecting deep tissue injury (DTI) before it is visible on a patient's skin, comprising: a) obtaining a set of SEM delta values at a location on the patient's skin at a predetermined frequency; b) applying to each of the SEM delta values of the obtained set a predetermined weight; c) calculating a first average SEM delta value of the N least-recent weighted SEM delta values; d) calculating a second average SEM delta value of the M most-recent weighted SEM delta values; e) comparing the difference between the first average and the second average SEM delta value with a predetermined threshold value; and f) determining that there is DTI at the location on the patient's skin when the difference is greater than the predetermined threshold value. In an aspect, the set of obtained SEM delta values comprises at least five SEM delta values each taken one day apart. In an aspect, the predetermined weights are in the range of 0 to 2. In an aspect, the predetermined weights monotonically increase with time. In an aspect, N>M. In an aspect, N is 4 and M is 2. In an aspect, the predetermined threshold value is a real number in the range of 0 to 1. In an aspect, the most recent SEM delta value is obtained by linear extrapolation of the K most-recent SEM delta values. In an aspect, K is 3. In an aspect, the predetermined threshold is 0.7.

In an aspect, a set of SEM delta values comprises a plurality of SEM delta values obtained at different times. In an aspect, a set of SEM delta values comprises a plurality of SEM delta values obtained at a predetermined frequency. In an aspect, a predetermined frequency is once a month, once every two weeks, once a week, once every five days, once every four days, once every three days, once every two days, once a day, once every 24 hours, once every 23 hours, once every 22 hours, once every 21 hours, once every 19 hours, once every 18 hours, once every 17 hours, once every 16 hours, once every 15 hours, once every 14 hours, once every 13 hours, once every 12 hours, once every 11 hours, once every 10 hours, once every 9 hours, once every 8 hours, once every 7 hours, once every 6 hours, once every 5 hours, once every 4 hours, once every 3 hours, once every 2 hours, once every hour, once every 60 min, once every 30 min, once every 15 min, once every 10 min, once every 5 min, once every 2 min. In an aspect, a set of SEM delta values comprises 10 SEM delta values obtain once every 24 hours. In an aspect, a set of SEM delta values comprises 9 SEM delta values obtain once every 24 hours. In an aspect, a set of SEM delta values comprises 8 SEM delta values obtain once every 24 hours. In an aspect, a set of SEM delta values comprises 7 SEM delta values obtain once every 24 hours. In an aspect, a set of SEM delta values comprises 6 SEM delta values obtain once every 24 hours. In an aspect, a set of SEM delta values comprises 5 SEM delta values obtain once every 24 hours. In an aspect, a set of SEM delta values comprises 4 SEM delta values obtain once every 24 hours. In an aspect, a set of SEM delta values comprises 3 SEM delta values obtain once every 24 hours. In an aspect, a set of SEM delta values comprises 2 SEM delta values obtain once every 24 hours.

In an aspect, a set of weights are applied to each SEM delta value in a set of SEM delta values. In an aspect, the set of weights are predetermined. In an aspect, a first predetermined weight is applied to the first SEM delta value in the set of SEM delta values. In an aspect, a second predetermined weight is applied to the second SEM delta value in the set of SEM delta values. In an aspect, the predetermined weight that is applied to a SEM delta value in the set of SEM delta values is determined by the time at which the SEM delta value is obtained, relative to the present time. In an aspect, a larger weight is applied to SEM delta values obtained more recently than to SEM delta values obtained at an earlier time. In an aspect, a smaller weight is applied to SEM delta values obtained at an earlier time compared to more recently obtained SEM delta values. In an aspect, the predetermined weights monotonically increase with time. In an aspect, the predetermined weights are in the range of 0 to 2. In an aspect, the predetermined weights are about 2, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1, or about 0.0. In an aspect, the predetermined weights are obtained from the results of a supervised learning algorithm. In an aspect, the predetermined weights are obtained from the results of a unsupervised learning algorithm.

In an aspect, an average SEM delta value is calculated from a set of SEM delta values. In an aspect, an average SEM delta value is calculated from a subset of SEM delta values. In an aspect, a first average SEM delta value is calculated from the N least-recent weighted SEM delta values. In an aspect, a second average SEM delta value is calculated from the M most-recent weighted SEM delta values. In an aspect, a first average SEM delta value is calculated from the 5 least-recent weighted SEM delta values, and a second average SEM delta value is calculated from the most-recent weighted SEM delta value. In an aspect, a first average SEM delta value is calculated from the 4 least-recent weighted SEM delta values, and a second average SEM delta value is calculated from the 2 most-recent weighted SEM delta values. In an aspect, a first average SEM delta value is calculated from the 3 least-recent weighted SEM delta values, and a second average SEM delta value is calculated from the 3 most-recent weighted SEM delta values. In an aspect, a first average SEM delta value is calculated from the 2 least-recent weighted SEM delta values, and a second average SEM delta value is calculated from the 4 most-recent weighted SEM delta values. In an aspect, a first average SEM delta value is calculated from the 1 least-recent weighted SEM delta values, and a second average SEM delta value is calculated from the 5 most-recent weighted SEM delta values. In an aspect, a second average SEM delta value is calculated from the most-recent weighted SEM delta value, and a first average SEM delta value is calculated from the next 5 most-recent weighted SEM delta values. In an aspect, a second average SEM delta value is calculated from the 2 most-recent weighted SEM delta values, and a first average SEM delta value is calculated from the next 4 most-recent weighted SEM delta values. In an aspect, a second average SEM delta value is calculated from the 3 most-recent weighted SEM delta values, and a first average SEM delta value is calculated from the next 3 most-recent weighted SEM delta values. In an aspect, a second average SEM delta value is calculated from the 4 most-recent weighted SEM delta values, and a first average SEM delta value is calculated from the next 2 most-recent weighted SEM delta values. In an aspect, a second average SEM delta value is calculated from the 5 most-recent weighted SEM delta values, and a first average SEM delta value is calculated from the next most-recent weighted SEM delta value. In an aspect, N+M=3. In an aspect, N+M=4. In an aspect, N+M=5. In an aspect, N+M=6. In an aspect, N+M=7. In an aspect, N+M=8. In an aspect, N+M=9. In an aspect, N+M=10. In an aspect, N=1, 2, 3, 4, 5, 6, 7, 8, 9, or 10. In an aspect, M=1, 2, 3, 4, 5, 6, 7, 8, 9, or 10. In an aspect, a plurality of SEM delta values associated with the patient is obtained periodically over a predetermined time interval preceding the risk assessment. In an aspect, the predetermined time interval preceding the risk assessment is 1 day, 2 days, 3 days, 4 days, 5 days, 6 days, 7 days, 8 days, 9 days or 10 days.

In an aspect, the difference between the first average and the second average SEM delta value is calculated. In an aspect, the difference between the first average and the second average SEM delta value is compared to a predetermined threshold. In an aspect, the predetermined threshold is zero. In an aspect, the predetermined threshold is a positive value. In an aspect, the predetermined threshold is a negative value. In an aspect, a positive difference between the first average and the second average SEM delta value indicates a deep tissue injury. In an aspect, a non-positive or zero difference between the first average and the second average SEM delta value indicates no deep tissue injury. In an aspect, a positive difference between the average of the most-recent and the average of the least-recent SEM delta values indicates a deep tissue injury. In an aspect, a non-positive or zero difference between the average of the most-recent and the average of the least-recent SEM delta values indicates no deep tissue injury. In an aspect, the predetermined threshold value is a real number in the range of 0 to 1. In an aspect, the predetermined threshold value is about 0.1, about 0.2, about 0.3, about 0.4, about 0.5, about 0.6, about 0.7, about 0.8, about 0.9, about 1.0. In an aspect, a DTI is determined to exist at the location on the patient's skin when the difference is greater than the predetermined threshold value.

### Methods of Training

In an aspect, the present disclosure provides a computer-implemented method for training a neural network for detection of deep tissue injury (DTI) before the injury is visible on a patient's skin, comprising: a) for each patient in a first plurality of patients who have been diagnosed with a DTI, obtaining a first set of sub-epidermal moisture (SEM) delta values at a location on the patient's skin at a predetermined frequency before and up to the formation of the DTI; b) for each patient in a second plurality of patients who have not been diagnosed with a DTI, obtaining a second set of SEM delta values at the same location on the patient's skin at the predetermined frequency; c) applying a set of weights to each of the SEM delta values of the first and second set of obtained SEM delta values; d) creating a training set comprising the first set of weighted SEM delta values and the second set of weighted SEM delta values of all patients in the first and second plurality of patients; and e) training the neural network using the training set.

In an aspect, the neural network is a single-layer neural network. In an aspect, the neural network is a multi-layer neural network. In an aspect, the neural network comprises at least one hidden layer, at least two hidden layers, at least three hidden layers, at least four hidden layers, or at least five hidden layers. In an aspect, the neural network uses a supervised learning algorithm. In an aspect, the neural network uses an unsupervised learning algorithm.

In an aspect, a first plurality of patients have been diagnosed with a DTI. In an aspect, for each patient in the first plurality of patients, a first set of SEM delta values is obtained at a location on the patient's skin. In an aspect, the first set of SEM delta values is obtained at a predetermined frequency before and up to the formation of the DTI. In an aspect, a second plurality of patients have not been diagnosed with a DTI. In an aspect, for each patient in the second plurality of patients, a second set of SEM delta values is obtained at a location on the patient's skin. In an aspect, the second set of SEM delta values is obtained at the same predetermined frequency as the first set of SEM delta values. In an aspect, a set of SEM delta values comprises a plurality of SEM delta values obtained at different times. In an aspect, a set of SEM delta values comprises a plurality of SEM delta values obtained at a predetermined frequency. In an aspect, a predetermined frequency is once a month, once every two weeks, once a week, once every five days, once every four days, once every three days, once every two days, once a day, once every 24 hours, once every 23 hours, once every 22 hours, once every 21 hours, once every 19 hours, once every 18 hours, once every 17 hours, once every 16 hours, once every 15 hours, once every 14 hours, once every 13 hours, once every 12 hours, once every 11 hours, once every 10 hours, once every 9 hours, once every 8 hours, once every 7 hours, once every 6 hours, once every 5 hours, once every 4 hours, once every 3 hours, once every 2 hours, once every hour, once every 60 minutes, once every 30 minutes, once every 15 minutes, once every 10 minutes, once every 5 minutes, once every 2 minutes, once every 1 minute, or once every 30 seconds. In an aspect, a set of SEM delta values comprises 10 SEM delta values obtain once every 24 hours. In an aspect, a set of SEM delta values comprises 9 SEM delta values obtain once every 24 hours. In an aspect, a set of SEM delta values comprises 8 SEM delta values obtain once every 24 hours. In an aspect, a set of SEM delta values comprises 7 SEM delta values obtain once every 24 hours. In an aspect, a set of SEM delta values comprises 6 SEM delta values obtain once every 24 hours. In an aspect, a set of SEM delta values comprises 5 SEM delta values obtain once every 24 hours. In an aspect, a set of SEM delta values comprises 4 SEM delta values obtain once every 24 hours. In an aspect, a set of SEM delta values comprises 3 SEM delta values obtain once every 24 hours. In an aspect, a set of SEM delta values comprises 2 SEM delta values obtain once every 24 hours. In an aspect, the location on the patient's skin is an area at risk of developing DTIs, including, but not limited to, the heel, the knees, the elbows, the sacrum, the thigh, the back of the head, the shoulders, the base of the spine, the buttocks, the toes, the ears, the hips, the legs, or the rib cage.

In an aspect, a set of weights are applied to each SEM delta value in a set of SEM delta values. In an aspect, the set of weights are random. In an aspect, a first random weight is applied to the first SEM delta value in the set of SEM delta values. In an aspect, a second random weight is applied to the second SEM delta value in the set of SEM delta values. In an aspect, the random weight that is applied to a SEM delta value in the set of SEM delta values is determined by the time at which the SEM delta value is obtained, relative to the present time. In an aspect, a larger random weight is applied to SEM delta values obtained more recently than to SEM delta values obtained at an earlier time. In an aspect, a smaller random weight is applied to SEM delta values obtained at an earlier time compared to more recently obtained SEM delta values. In an aspect, the random weights monotonically increase with time.

In an aspect, a training set comprising the first set of weighted SEM delta values and the second set of weighted SEM delta values of all patients in the first and second plurality of patients is created. In an aspect, the neural network is trained using the training set.

In an aspect, the method further comprises: a) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; b) creating a training set comprising a first set of difference values in the first plurality of patients and a second set of difference values in the second plurality of patients; and c) training the neural network using the training set.

In an aspect, an average SEM delta value is calculated from a set of SEM delta values. In an aspect, an average SEM delta value is calculated from a subset of SEM delta values. In an aspect, a first average SEM delta value is calculated from the N least-recent weighted SEM delta values. In an aspect, a second average SEM delta value is calculated from the M most-recent weighted SEM delta values. In an aspect, a first average SEM delta value is calculated from the 5 least-recent weighted SEM delta values, and a second average SEM delta value is calculated from the most-recent weighted SEM delta value. In an aspect, a first average SEM delta value is calculated from the 4 least-recent weighted SEM delta values, and a second average SEM delta value is calculated from the 2 most-recent weighted SEM delta values. In an aspect, a first average SEM delta value is calculated from the 3 least-recent weighted SEM delta values, and a second average SEM delta value is calculated from the 3 most-recent weighted SEM delta values. In an aspect, a first average SEM delta value is calculated from the 2 least-recent weighted SEM delta values, and a second average SEM delta value is calculated from the 4 most-recent weighted SEM delta values. In an aspect, a first average SEM delta value is calculated from the 1 least-recent weighted SEM delta values, and a second average SEM delta value is calculated from the 5 most-recent weighted SEM delta values. In an aspect, a second average SEM delta value is calculated from the most-recent weighted SEM delta value, and a first average SEM delta value is calculated from the next 5 most-recent weighted SEM delta values. In an aspect, a second average SEM delta value is calculated from the 2 most-recent weighted SEM delta values, and a first average SEM delta value is calculated from the next 4 most-recent weighted SEM delta values. In an aspect, a second average SEM delta value is calculated from the 3 most-recent weighted SEM delta values, and a first average SEM delta value is calculated from the next 3 most-recent weighted SEM delta values. In an aspect, a second average SEM delta value is calculated from the 4 most-recent weighted SEM delta values, and a first average SEM delta value is calculated from the next 2 most-recent weighted SEM delta values. In an aspect, a second average SEM delta value is calculated from the 5 most-recent weighted SEM delta values, and a first average SEM delta value is calculated from the next most-recent weighted SEM delta value. In an aspect, a plurality of SEM delta values associated with the patient is obtained periodically over a predetermined time interval preceding the risk assessment. In an aspect, the predetermined time interval preceding the risk assessment is 1 day, 2 days, 3 days, 4 days, 5 days, 6 days, 7 days, 8 days, 9 days or 10 days.

In an aspect, the difference between the first average and the second average SEM delta value is calculated. In an aspect, the difference between the first average and the second average SEM delta value is compared to a threshold. In an aspect, the threshold is zero. In an aspect, the threshold is a positive value. In an aspect, the threshold is a negative value. In an aspect, a positive difference between the first average and the second average SEM delta value indicates a deep tissue injury. In an aspect, a non-positive or zero difference between the first average and the second average SEM delta value indicates no deep tissue injury. In an aspect, a positive difference between the average of the most-recent and the average of the least-recent SEM delta values indicates a deep tissue injury. In an aspect, a non-positive or zero difference between the average of the most-recent and the average of the least-recent SEM delta values indicates no deep tissue injury. In an aspect, the threshold value is a real number in the range of 0 to 1. In an aspect, the threshold value is about 0.1, about 0.2, about 0.3, about 0.4, about 0.5, about 0.6, about 0.7, about 0.8, about 0.9, about 1.0. In an aspect, a DTI is determined to exist at the location on the patient's skin when the difference is greater than the predetermined threshold value.

In an aspect, a training set comprising the first set of difference values in the first plurality of patients and a second set of difference values in the second plurality of patients is created. In an aspect, the neural network is trained using the training set.

In an aspect, the trained neural network outputs a set of optimized weights, comprising an optimized weight for each timepoint before the formation of a DTI. In an aspect, the optimized weights monotonically increase with time. In an aspect, the optimized weights are in the range of 0 to 2. In an aspect, the optimized weights are about 2, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1, or about 0.0. In an aspect, the trained neural network outputs an optimized threshold. In an aspect, the optimized threshold value is about 0.1, about 0.2, about 0.3, about 0.4, about 0.5, about 0.6, about 0.7, about 0.8, about 0.9, about 1.0.

In an aspect, the present disclosure provides a computer-implemented method for training a neural network for detection of deep tissue injury (DTI) before the injury is visible on a patient's skin, comprising: a) for each patient in a first plurality of patients who experience a DTI, obtaining a first set of sub-epidermal moisture (SEM) delta values at a location on the patient's skin at a predetermined frequency before and up to the formation of the DTI; b) for each patient in a second plurality of patients who do not experience a DTI, obtaining a second set of SEM delta values at the same location on the patient's skin at the predetermined frequency; c) applying a set of weights to each of the SEM delta values of the first and second set of obtained SEM delta values; d) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; e) creating a training set comprising a first set of difference values in the first plurality of patients and a second set of difference values in the second plurality of patients; and f) training the neural network using the training set.

In an aspect, the neural network is a single-layer neural network. In an aspect, the neural network is a multi-layer neural network. In an aspect, the neural network comprises at least one hidden layer, at least two hidden layers, at least three hidden layers, at least four hidden layers, or at least five hidden layers. In an aspect, the neural network uses a supervised learning algorithm. In an aspect, the neural network uses an unsupervised learning algorithm.

In an aspect, a first plurality of patients have been diagnosed with a DTI. In an aspect, for each patient in the first plurality of patients, a first set of SEM delta values is obtained at a location on the patient's skin. In an aspect, the first set of SEM delta values is obtained at a predetermined frequency before and up to the formation of the DTI. In an aspect, a second plurality of patients have not been diagnosed with a DTI. In an aspect, for each patient in the second plurality of patients, a second set of SEM delta values is obtained at a location on the patient's skin. In an aspect, the second set of SEM delta values is obtained at the same predetermined frequency as the first set of SEM delta values. In an aspect, a set of SEM delta values comprises a plurality of SEM delta values obtained at different times. In an aspect, a set of SEM delta values comprises a plurality of SEM delta values obtained at a predetermined frequency. In an aspect, a predetermined frequency is once a month, once every two weeks, once a week, once every five days, once every four days, once every three days, once every two days, once a day, once every 24 hours, once every 23 hours, once every 22 hours, once every 21 hours, once every 19 hours, once every 18 hours, once every 17 hours, once every 16 hours, once every 15 hours, once every 14 hours, once every 13 hours, once every 12 hours, once every 11 hours, once every 10 hours, once every 9 hours, once every 8 hours, once every 7 hours, once every 6 hours, once every 5 hours, once every 4 hours, once every 3 hours, once every 2 hours, once every hour, once every 60 min, once every 30 min, once every 15 min, once every 10 min, once every 5 min, once every 2 min. In an aspect, a set of SEM delta values comprises 10 SEM delta values obtain once every 24 hours. In an aspect, a set of SEM delta values comprises 9 SEM delta values obtain once every 24 hours. In an aspect, a set of SEM delta values comprises 8 SEM delta values obtain once every 24 hours. In an aspect, a set of SEM delta values comprises 7 SEM delta values obtain once every 24 hours. In an aspect, a set of SEM delta values comprises 6 SEM delta values obtain once every 24 hours. In an aspect, a set of SEM delta values comprises 5 SEM delta values obtain once every 24 hours. In an aspect, a set of SEM delta values comprises 4 SEM delta values obtain once every 24 hours. In an aspect, a set of SEM delta values comprises 3 SEM delta values obtain once every 24 hours. In an aspect, a set of SEM delta values comprises 2 SEM delta values obtain once every 24 hours. In an aspect, the location on the patient's skin is an area at risk of developing DTIs, including, but not limited to, the heel, the knees, the elbows, the sacrum, the thigh, the back of the head, the shoulders, the base of the spine, the buttocks, the toes, the ears, the hips, the legs, or the rib cage.

In an aspect, a set of weights are applied to each SEM delta value in a set of SEM delta values. In an aspect, the set of weights are random. In an aspect, a first random weight is applied to the first SEM delta value in the set of SEM delta values. In an aspect, a second random weight is applied to the second SEM delta value in the set of SEM delta values. In an aspect, the random weight that is applied to a SEM delta value in the set of SEM delta values is determined by the time at which the SEM delta value is obtained, relative to the present time. In an aspect, a larger random weight is applied to SEM delta values obtained more recently than to SEM delta values obtained at an earlier time. In an aspect, a smaller random weight is applied to SEM delta values obtained at an earlier time compared to more recently obtained SEM delta values. In an aspect, the random weights monotonically increase with time.

In an aspect, a training set comprising the first set of weighted SEM delta values and the second set of weighted SEM delta values of all patients in the first and second plurality of patients is created. In an aspect, the neural network is trained using the training set.

In an aspect, an average SEM delta value is calculated from a set of SEM delta values. In an aspect, an average SEM delta value is calculated from a subset of SEM delta values. In an aspect, a first average SEM delta value is calculated from the N least-recent weighted SEM delta values. In an aspect, a second average SEM delta value is calculated from the M most-recent weighted SEM delta values. In an aspect, a first average SEM delta value is calculated from the 5 least-recent weighted SEM delta values, and a second average SEM delta value is calculated from the most-recent weighted SEM delta value. In an aspect, a first average SEM delta value is calculated from the 4 least-recent weighted SEM delta values, and a second average SEM delta value is calculated from the 2 most-recent weighted SEM delta values. In an aspect, a first average SEM delta value is calculated from the 3 least-recent weighted SEM delta values, and a second average SEM delta value is calculated from the 3 most-recent weighted SEM delta values. In an aspect, a first average SEM delta value is calculated from the 2 least-recent weighted SEM delta values, and a second average SEM delta value is calculated from the 4 most-recent weighted SEM delta values. In an aspect, a first average SEM delta value is calculated from the 1 least-recent weighted SEM delta values, and a second average SEM delta value is calculated from the 5 most-recent weighted SEM delta values. In an aspect, a second average SEM delta value is calculated from the most-recent weighted SEM delta value, and a first average SEM delta value is calculated from the next 5 most-recent weighted SEM delta values. In an aspect, a second average SEM delta value is calculated from the 2 most-recent weighted SEM delta values, and a first average SEM delta value is calculated from the next 4 most-recent weighted SEM delta values. In an aspect, a second average SEM delta value is calculated from the 3 most-recent weighted SEM delta values, and a first average SEM delta value is calculated from the next 3 most-recent weighted SEM delta values. In an aspect, a second average SEM delta value is calculated from the 4 most-recent weighted SEM delta values, and a first average SEM delta value is calculated from the next 2 most-recent weighted SEM delta values. In an aspect, a second average SEM delta value is calculated from the 5 most-recent weighted SEM delta values, and a first average SEM delta value is calculated from the next most-recent weighted SEM delta value.

In an aspect, the difference between the first average and the second average SEM delta value is calculated. In an aspect, the difference between the first average and the second average SEM delta value is compared to a threshold. In an aspect, the threshold is zero. In an aspect, the threshold is a positive value. In an aspect, the threshold is a negative value. In an aspect, a positive difference between the first average and the second average SEM delta value indicates a deep tissue injury. In an aspect, a non-positive or zero difference between the first average and the second average SEM delta value indicates no deep tissue injury. In an aspect, a positive difference between the average of the most-recent and the average of the least-recent SEM delta values indicates a deep tissue injury. In an aspect, a non-positive or zero difference between the average of the most-recent and the average of the least-recent SEM delta values indicates no deep tissue injury. In an aspect, the threshold value is a real number in the range of 0 to 1. In an aspect, the threshold value is about 0.1, about 0.2, about 0.3, about 0.4, about 0.5, about 0.6, about 0.7, about 0.8, about 0.9, about 1.0. In an aspect, a DTI is determined to exist at the location on the patient's skin when the difference is greater than the predetermined threshold value.

In an aspect, a training set comprising the first set of difference values in the first plurality of patients and a second set of difference values in the second plurality of patients is created. In an aspect, the neural network is trained using the training set.

In an aspect, the trained neural network outputs: a) a set of optimized weights, comprising an optimized weight for each timepoint before the formation of a DTI, where the optimized weights monotonically increase with time; b) an optimized threshold; c) the value of N; and d) the value of M. In an aspect, the set of optimized weights and the optimized threshold are used to predict the occurrence of deep tissue injury. In an aspect, the optimized weights monotonically increase with time. In an aspect, the optimized weights are in the range of 0 to 2. In an aspect, the optimized weights are about 2, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, about 0.1, or about 0.0. In an aspect, the trained neural network outputs an optimized threshold. In an aspect, the optimized threshold value is about 0.1, about 0.2, about 0.3, about 0.4, about 0.5, about 0.6, about 0.7, about 0.8, about 0.9, about 1.0. In an aspect, N+M =3. In an aspect, N+M =4. In an aspect, N+M =5. In an aspect, N+M =6. In an aspect, N+M =7. In an aspect, N+M =8. In an aspect, N+M =9. In an aspect, N+M =10. In an aspect, N =1, 2, 3, 4, 5, 6, 7, 8, 9, or 10. In an aspect, M =1, 2, 3, 4, 5, 6, 7, 8, 9, or 10.

In an aspect, the set of optimized weights and the optimized threshold are used to predict the occurrence of deep tissue injury.

### Computer Readable Media (Software)

Any of the aforementioned methods of present disclosure may be implemented as computer program processes that are specified as a set of instructions recorded on a non-transitory computer-readable storage medium or computer-readable medium (CRM).

In an aspect, the present disclosure provides a non-transitory computer-readable storage medium comprising one or more programs for execution by one or more processors of a device, the one or more programs comprising instructions which, when executed by the one or more processors, cause the device to perform any of the methods disclosed herein.

Also provided herein is a non-transitory computer-readable storage medium comprising a report generated from performing any of the methods disclosed herein.

Examples of computer-readable storage media include random access memory (RAM), read-only memory (ROM), read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In some embodiments, the computer-readable storage medium is a solid-state device, a hard disk, a CD-ROM, or any other non-volatile computer-readable storage medium.

The computer-readable storage media can store a set of computer-executable instructions (e.g., a “computer program”) that is executable by at least one processing unit and includes sets of instructions for performing various operations.

A computer program (also known as a program, software, software application, script, or code) can be written in any form of programming language, including compiled or interpreted languages, declarative or procedural languages, and it can be deployed in any form, including as a standalone program or as a module, component, or subroutine, object, or other component suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms or portions of code). A computer program can be deployed to be executed on one computer or on multiple computers that are located at one site or distributed across multiple sites and interconnected by a communication network. Examples of computer programs or computer code include machine code, such as is produced by a compiler, and files including higher-level code that are executed by a computer, an electronic component, or a microprocessor using an interpreter.

In some embodiments, multiple software aspects of the subject disclosure can be implemented as sub-parts of a larger program while remaining distinct software aspects of the subject disclosure. In some implementations, multiple software aspects can also be implemented as separate programs. Any combination of separate programs that together implement a software aspect described here is within the scope of the subject disclosure. In some implementations, the software programs, when installed to operate on one or more electronic systems, define one or more specific machine implementations that execute and perform the operations of the software programs.

The present disclosure provides for, and includes, a non-transitory computer readable medium for identifying DTI tissue. The non-transitory computer readable medium may comprise instructions stored thereon, that when executed on a processor, may perform the steps of: a) obtaining a set of SEM delta values at a location on the patient's skin at a predetermined frequency; b) applying to each of the SEM delta values of the obtained set a predetermined weight; c) calculating a first average SEM delta value of the N least-recent weighted SEM delta values; d) calculating a second average SEM delta value of the M most-recent weighted SEM delta values; e) comparing the difference between the first average and the second average SEM delta value with a predetermined threshold value; and f) determining that there is DTI at the location on the patient's skin when the difference is greater than the predetermined threshold value. In an aspect, the set of obtained SEM delta values comprises at least five SEM delta values each taken one day apart. In an aspect, the predetermined weights are in the range of 0 to 2. In an aspect, the predetermined weights monotonically increase with time. In an aspect, N>M. In an aspect, N is 4 and M is 2. In an aspect, the predetermined threshold value is a real number in the range of 0 to 1. In an aspect, the most recent SEM delta value is obtained by linear extrapolation of the K most-recent SEM delta values. In an aspect, K is 3. In an aspect, the predetermined threshold is 0.7. In an aspect, the location on the patient's skin is selected from the group consisting of the back of the head, the sacrum, the shoulder, the elbow, the lower back, the tailbone, the buttock, the hip, the inner knee, the heel, and a combination thereof.

The present disclosure provides for, and includes, a non-transitory computer readable medium for identifying DTI tissue. The non-transitory computer readable medium may comprise instructions stored thereon, that when executed on a processor, may perform the steps of: a) for each patient in a first plurality of patients who have been diagnosed with a DTI, obtaining a first set of sub-epidermal moisture (SEM) delta values at a location on the patient's skin at a predetermined frequency before and up to the formation of the DTI; b) for each patient in a second plurality of patients who have not been diagnosed with a DTI, obtaining a second set of SEM delta values at the same location on the patient's skin at the predetermined frequency; c) applying a set of weights to each of the SEM delta values of the first and second set of obtained SEM delta values; d) creating a training set comprising the first set of weighted SEM delta values and the second set of weighted SEM delta values of all patients in the first and second plurality of patients; and e) training the neural network using the training set. In an aspect, the trained neural network outputs a set of optimized weights, comprising an optimized weight for each timepoint before the formation of a DTI, and where the optimized weights monotonically increase with time. In an aspect, the method further comprises the steps of: a) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; b) creating a training set comprising a first set of difference values in the first plurality of patients and a second set of difference values in the second plurality of patients; and c) training the neural network using the training set. In an aspect, the predetermined frequency is once a day. In an aspect, the first and second set of SEM delta values comprises at least six SEM delta values each taken one day apart. In an aspect, N+M=6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof .

The present disclosure provides for, and includes, a non-transitory computer readable medium for identifying DTI tissue. The non-transitory computer readable medium may comprise instructions stored thereon, that when executed on a processor, may perform the steps of: a) for each patient in a first plurality of patients who experience a DTI, obtaining a first set of sub-epidermal moisture (SEM) delta values at a location on the patient's skin at a predetermined frequency before and up to the formation of the DTI; b) for each patient in a second plurality of patients who do not experience a DTI, obtaining a second set of SEM delta values at the same location on the patient's skin at the predetermined frequency; c) applying a set of weights to each of the SEM delta values of the first and second set of obtained SEM delta values; d) for each patient in the first and second plurality of patients, i) calculating a first average SEM delta value of the N least-recent weighted SEM delta values in the set of SEM delta values; ii) calculating a second average SEM delta value of the M most-recent weighted SEM delta values in the set of SEM delta values; iii) calculating a difference value between the first average and the second average SEM delta value; and iv) comparing the difference between the first average and the second average SEM delta value with a threshold value; e) creating a training set comprising a first set of difference values in the first plurality of patients and a second set of difference values in the second plurality of patients; and f) training the neural network using the training set. In an aspect, the trained neural network outputs a) a set of optimized weights, comprising an optimized weight for each timepoint before the formation of a DTI, where the optimized weights monotonically increase with time; b) an optimized threshold; c) the value of N; and d) the value of M. In an aspect, the predetermined frequency is once a day. In an aspect, the first and second set of SEM delta values comprises at least six SEM delta values each taken one day apart. In an aspect, N+M=6. In an aspect, N=4 and M=2. In an aspect, the location on the patient's skin is selected from the group consisting of the back of the head, the sacrum, the shoulder, the elbow, the lower back, the tailbone, the buttock, the hip, the inner knee, the heel, and a combination thereof . In an aspect, the set of optimized weights and the optimized threshold are used to predict the occurrence of deep tissue injury.

In an aspect, the location on the patient's skin may be a bony prominence. In an aspect, the location on the patient's skin is a sternum, sacrum, a heel, a scapula, an elbow, an ear, or other fleshy tissue. In an aspect, one SEM value is measured at the anatomical site. In an aspect, an average SEM value at the location on the patient's skin is obtained from two, three, four, five, six, seven, eight, nine, ten, or more than ten SEM values measured at the location.

Systems

Any of the aforementioned methods and devices of the present disclosure may be implemented in systems, or a collection of devices.

The present disclosure provides a system for predicting DTIs. In an aspect, the system comprises a device capable of making SEM measurements, including but not limited to that described in U.S. Pat. No. 9,398,879B2. In an aspect, the system comprises a device capable of making SEM measurements including but not limited to that described in U.S. Pat. No. 10,182,740B2. Both U.S. Pat. Nos. 9,398,879B2 and 10,182,740B2 are incorporated herein by reference in their entireties. In an aspect, the system comprises a device capable of making SEM measurements, including but not limited to the SEM Scanner Model 200 (Bruin Biometrics, LLC, Los Angeles, Calif.). In an aspect, the system comprises a device capable of making biocapacitance measurements. In an aspect, the system is configured to perform the methods described herein. In an aspect, the system comprises a processor. In an aspect, the system comprises a non-transitory computer-readable medium electronically coupled to the processor, and comprising instructions stored thereon that, when executed on the processor, perform the steps of the methods described herein.

In an aspect, the system comprises a device capable of making SEM measurements or biocapacitance measurements, the device comprising a coverlay. In an aspect, the coverlay may be a double-sided, copper-clad laminate and an all-polyimide composite of a polyimide film bonded to copper foil. In an aspect, the coverlay may comprise Pyralux 5 mil FR0150. Without being limited by theory, the use of this coverlay may avoid parasitic charges naturally present on the skin surface from interfering with the accuracy and precision of SEM measurements.

In an aspect, the methods disclosed herein are performed in one or more electronic devices, including: one or more processors; a memory; and one or more programs, where the one or more programs comprising instructions which, when executed by the one or more processors, cause the device to perform the method of any one of the preceding embodiments. Examples of devices further include but are not limited to, a computer, a tablet personal computer, a personal digital assistant, and a cellular telephone.

In some embodiments, the electronic device may further include one or more displays. In some embodiments, the electronic device includes one or more displays to present a report generated from performing the method of any one of the preceding embodiments.

In some embodiments, the electronic device may be a server computer, a client computer, a personal computer (PC), a user device, a tablet PC, a laptop computer, a personal digital assistant (PDA), a cellular telephone, or any machine capable of executing a set of instructions, sequential or otherwise, that specify actions to be taken by that machine. In some embodiments, the electronic device may further include keyboard and pointing devices, touch devices, barcode scanners, display devices, and network devices.

To provide for interaction with a user, implementations of the subject matter described in this specification can be implemented on a computer having a display device described herein for displaying information to the user and a virtual or physical keyboard and a pointing device, such as a finger, pencil, mouse or a trackball, by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speed, or tactile input.

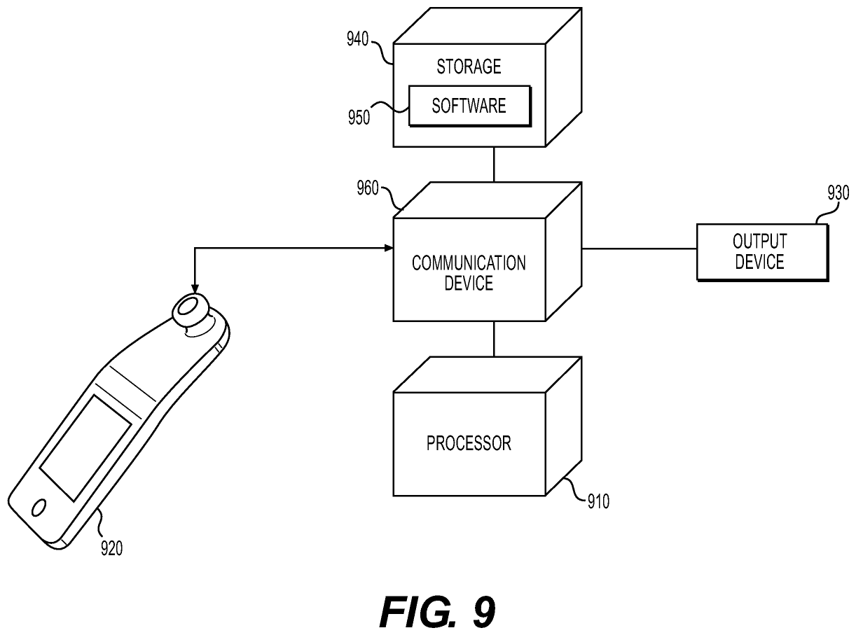

FIG. 9 illustrates an exemplary system 900 in accordance with one embodiment. In an aspect, system 900 comprises a host computer connected to a network. In an aspect, system 900 comprises a client computer or a server. In an aspect, system 900 can comprise any suitable type of microprocessor-based device, such as a personal computer, workstation, server or handheld computing device (portable electronic device) such as a phone or tablet. As shown in FIG. 9, in an aspect, system 900 comprises one or more of a processor 910, an input device 920, an output device 930, a storage medium 940, and a communication device 960. Input device 920 and output device 930 can generally correspond to those described above, and can be connectable or integrated with the computer.

In an aspect, input device 920 is any suitable device that provides input, such as a touch screen, keyboard or keypad, mouse, or voice-recognition device. In an aspect, input device 920 is an SEM measurement device. In an aspect, output device 930 is suitable device that provides output, such as a display, a touch screen, haptics device, or speaker.

In an aspect, storage 940 is any suitable device that provides storage, such as an electrical, magnetic or optical memory, including but not limited to a RAM, ROM, cache, hard drive, and removable storage disk. In an aspect, communication device 960 include any suitable device capable of transmitting and receiving signals over a network, including but not limited to a network interface chip or device, a router, a wireless card, and a Bluetooth signal emitter and receiver. In an aspect, the components of the system 900 are individually connected in any suitable manner, including but not limited to a physical bus, wires, wirelessly, Bluetooth connections, infrared, and radio signals.

In an aspect, software 950, which can be stored in storage 940 and executed by processor 910, includes, for example, the programming that embodies the functionality of the present disclosure (e.g., as embodied in the devices as described above). In an aspect, software 950 is stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In an aspect, a computer-readable storage medium is any medium, such as storage 940, that can contain or store programming for use by or in connection with an instruction execution system, apparatus, or device.

In an aspect, software 950 is propagated within any transport medium for use by or in connection with an instruction execution system, apparatus, or device, such as those described herein, that can fetch instructions associated with the software from the instruction execution system, apparatus, or device and execute the instructions. In an aspect, a transport medium is any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

In an aspect, system 900 may be connected to a network, which is any suitable type of interconnected communication system. In an aspect, the network can implement any suitable communications protocol and can be secured by any suitable security protocol. In an aspect, the network comprises network links of any suitable arrangement that can implement the transmission and reception of network signals, including but not limited to wireless network connections, T1 or T3 lines, cable networks, DSL, and telephone lines.

In an aspect, system 900 implements an operating system suitable for operating on the network. In an aspect, software 950 is written in any suitable programming language, including but not limited to C, C++, Java or Python. In an aspect, application software embodying the functionality of the present disclosure can be deployed in different configurations, such as in a client/server arrangement or through a Web browser as a Web-based application or Web service, for example.

### Methods of Preventing and Treating DTI Formation

Methods according to the present disclosure may prevent DTI formation in a patient. These prevention methods may be applied to patients prior to a diagnosis of a DTI, based on the methods described herein. In an aspect, at least five SEM delta measurements may be obtained around a location on a patient's skin using an apparatus in accordance with the present disclosure. In an aspect, linear interpolation is used to obtain the value of the most-recent SEM delta value. In an aspect, direct measurement is used to obtain the value of the most-recent SEM delta value. In an aspect, the methods disclosed herein are used to determine if there is deep tissue injury at the location on the patient's skin. In an aspect, preventative measures are taken if the methods disclosed herein determines that there is deep tissue injury at the location. In an aspect, the location on the patient's skin is selected from the group consisting of the back of the head, the sacrum, the shoulder, the elbow, the lower back, the tailbone, the buttock, the hip, the inner knee, the heel, and a combination thereof.

In an aspect, a preventative measure against DTI formation may be performed on a patient at the time of hospital intake of the patient, immediately after a surgical procedure, prior to hospital discharge of the patient, or any combination of the foregoing. The surgical procedure may be invasive or non-invasive, but may require a patient to remain in the same position for some period of time, such as, for example, at least 1 hour.

In an aspect, a preventative measure may be selected from the group consisting of turning and repositioning the patient at least every two hours; protecting bony prominence with padding; setting a specific turning and repositioning schedule; providing wedge devices for lateral positioning; providing a pressure redistribution support surface; managing moisture, nutrition, friction, and shear; reducing pressure of bony prominence; increasing frequency of turning, including small shifts of weight; reassessing SEM level every two hours; and any combination thereof

Methods according to the present disclosure may be used to treat PI in a patient that is detected by the methods described herein. These treatment methods may be applied to patients after detection of a DTI, based on the methods described herein. In an aspect, at least five SEM delta measurements are obtained around a location on a patient's skin using an apparatus in accordance with the present disclosure. In an aspect, linear interpolation is used to obtain the value of the most-recent SEM delta value. In an aspect, direct measurement is used to obtain the value of the most-recent SEM delta value. In an aspect, the methods disclosed herein are used to determine if there is deep tissue injury at the location on the patient's skin. In an aspect, treatment methods are performed if the methods disclosed herein determines that there is deep tissue injury at the location.

Methods according to the present disclosure may treat a pre-existing DTI. In an aspect, a treatment method is selected from the group consisting of protecting the skin; removing excessive pressure points; managing exudates; closing and re-growing damaged skin; keeping the wound clean; reducing wound size; and any combination thereof.

Having now generally described the invention, the same will be more readily understood through reference to the following examples that are provided by way of illustration, and are not intended to be limiting of the present disclosure, unless specified.

### EXAMPLES

### Example 1

### Obtaining SEM Delta Values of Patients With and Without DTI on the Heel

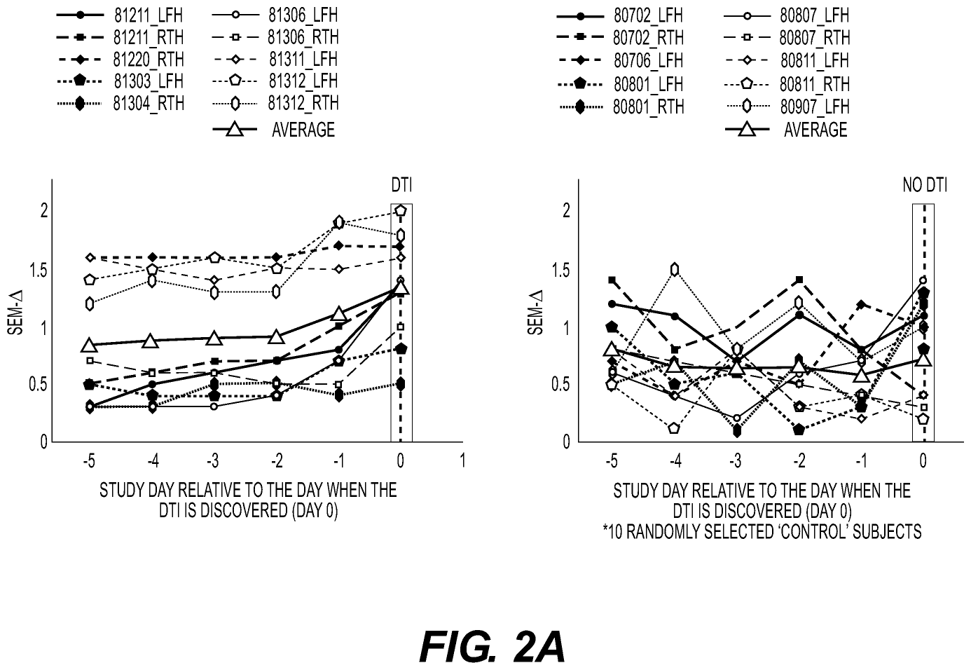

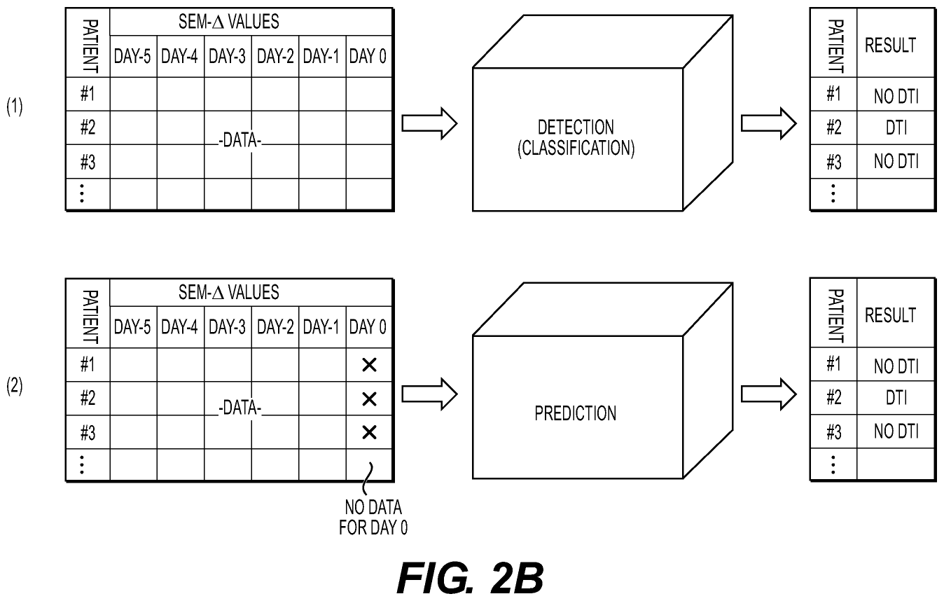

A blinded clinical trial is conducted to measure the SEM delta values of patients with skin and tissue at increased risk of developing pressure injuries. SEM delta values are measured in patients on the heel over time, at a frequency of once a day. Patients are visually assessed daily for deep tissue injury (DTI). As shown in FIG. 2A, patient data is categorized into two groups, patients who are diagnosed with DTI (left panel) and patients who are yet to be diagnosed with DTI (right panel). For patients who are diagnosed with DTI, the SEM delta values data in the 5 days leading up to diagnosis are aligned to the day of diagnosis (Day were aligned to the day of diagnosis (Day 0). Data from patients are used to build a classification algorithm to detect and predict formation of DTIs, as shown in FIG. 2B.

### Example 2

### Averaging Most-Recent and Least-Recent SEM Delta Values

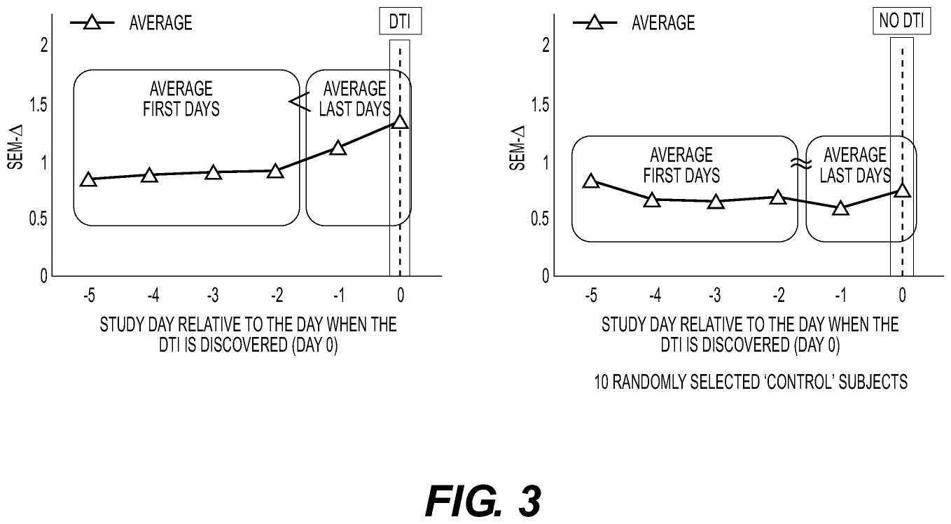

As shown in FIG. 3, the average SEM delta values in the days preceding the formation of a DTI are greater than the average SEM delta values in the days prior. On the other hand, when no DTI is diagnosed, the average SEM delta values are similar across all days.

Accordingly, the average SEM delta values of the days immediately preceding diagnosis of DTI and that of the days not immediately preceding diagnosis can be used as a criteria for classification. In particular, the following inequalities is used to classify:
Average(SEM delta_{last_days})−Average(SEM delta_{first_days})>0
Average(SEM delta_{last_days})−Average(SEM delta_{first_days})>threshold

When the obtained SEM delta values satisfy the above inequalities, the model assumes that a DTI is formed. This is then compared to the actual data of whether a DTI is diagnosed to determine a true positive rate (TPR) and a false positive rate (FPR).

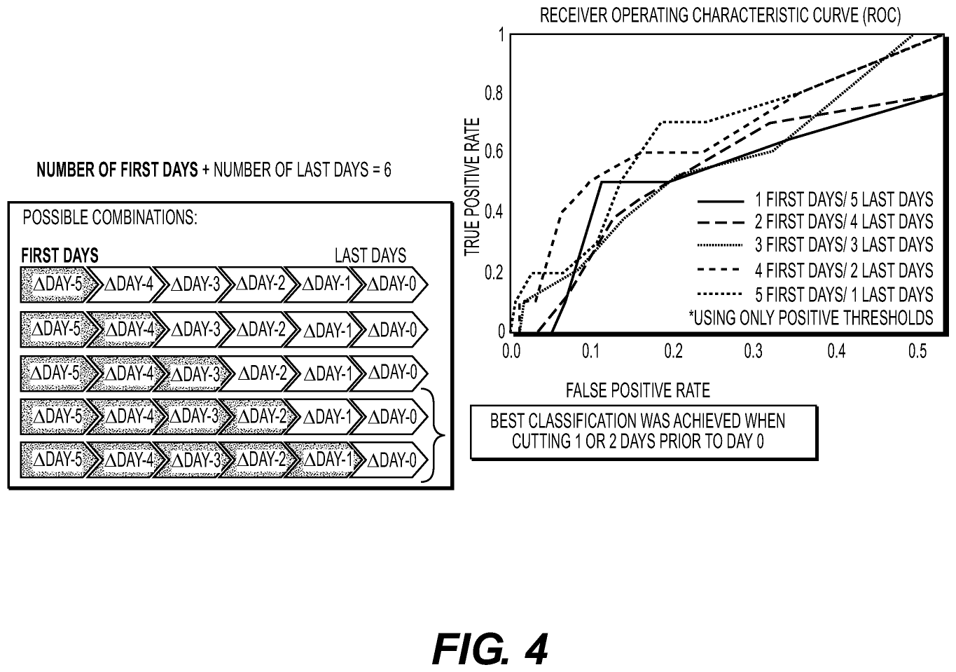

As shown in FIG. 4, when there are 6 data points, there are 5 possible ways to divide the data to determine most-recent (last days) and least-recent data (first days). The most-recent SEM delta value (or last days) could be an average of the most recent day, the two most recent days, the three most recent days, the four most recent days, and the five most recent days. Conversely, the least-recent SEM delta value (or first days) could be an average of the least recent day, the two least recent days, the three least recent days, the fourth least recent days, and the five least recent days.

The best classification, as determined by the highest true positive rate and the lowest false positive rate, is achieved using 2 most-recent (last days) SEM delta values with 4 least-recent (first days) SEM delta values.

### Example 3

### Determining Weights and Thresholds

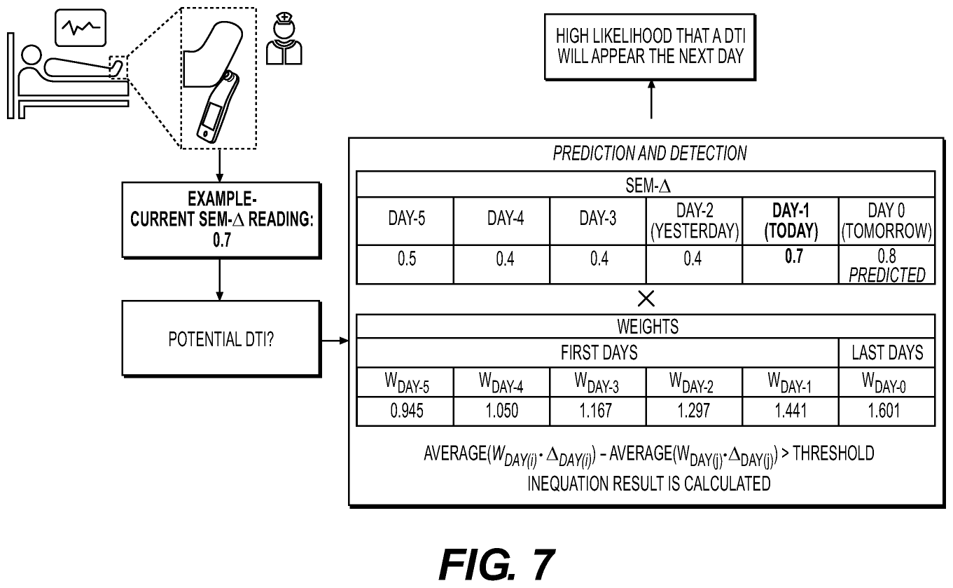

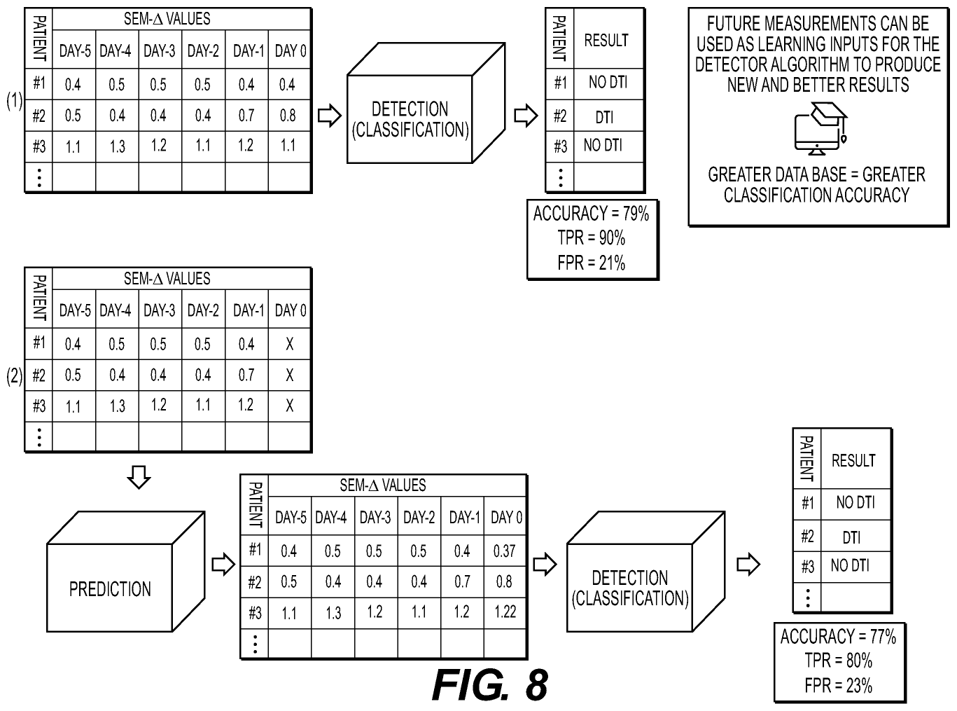

Before calculating the average SEM delta values, the days are assigned with monotonically increasing weights (wᵢ) representing the different influence of each sampling day on the result. Thus, the inequality becomes:
Average(w_{day(i)}×SEM delta_{day(i)}−Average(w_{day(j)}×SEM delta_{day(j)})>threshold
where i=last days and j=first days, as shown in FIG. 7.

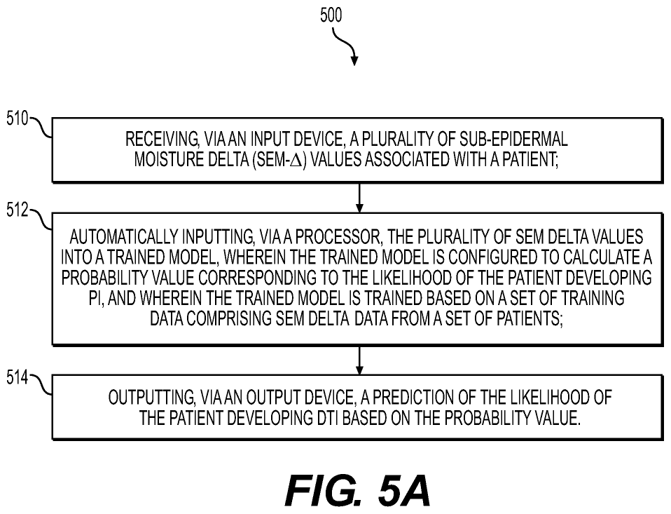

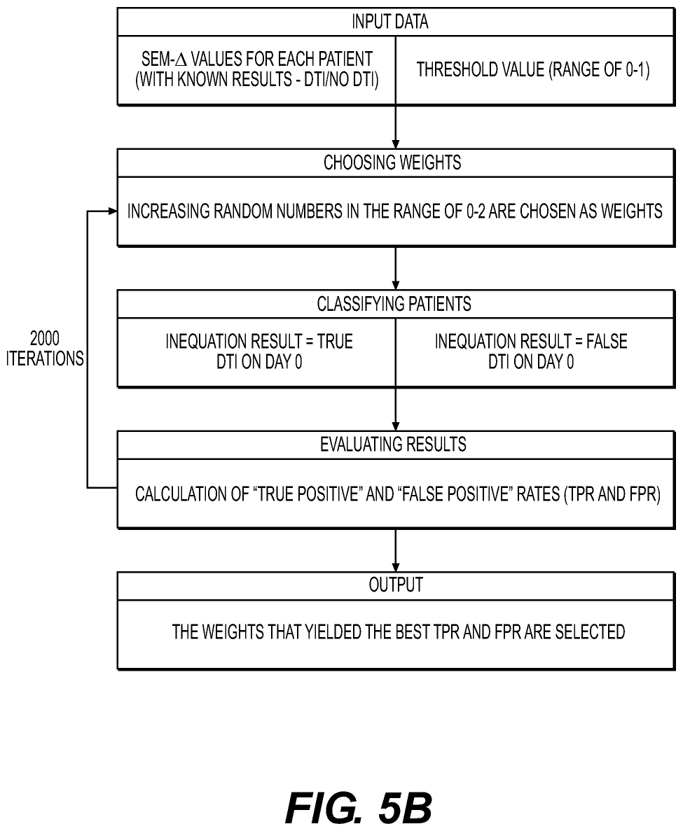

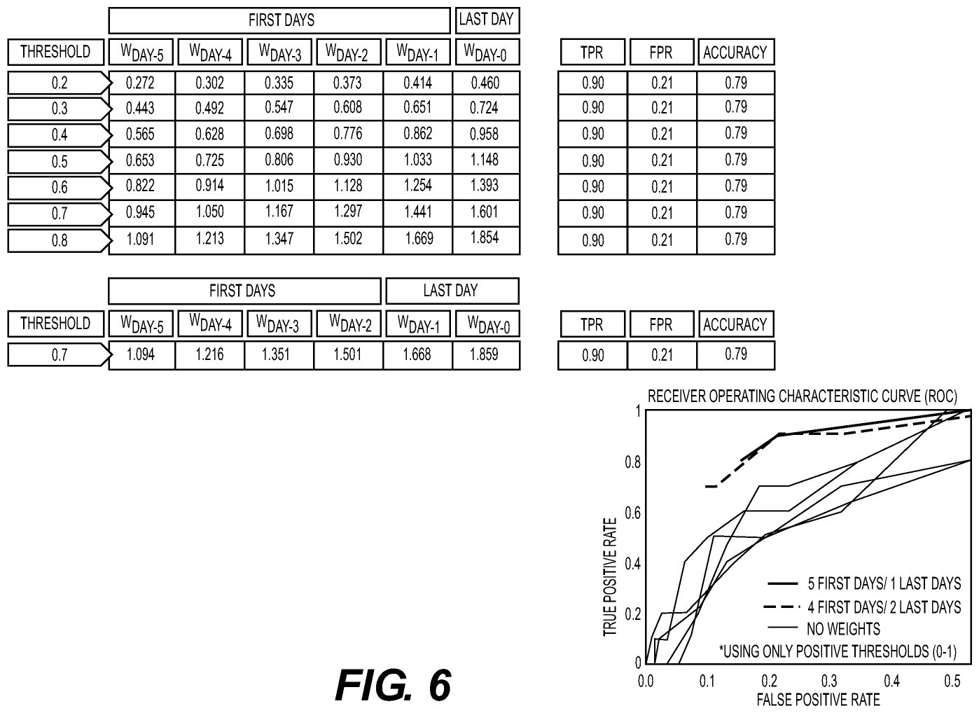

An optimization algorithm is used to find the optimal set of weights that provides the best classification performance, as determined by the true positive rate and the false positive rates. In particular, the algorithm sought to minimize (1−TPR+FPR)→0 with the constraint that TPR»FPR. FIG. 5 depicts the flowchart of the steps of the algorithm, while FIG. 6 shows the weights and threshold that yields the best classification results.

### Example 4

### Predicting Formation of DTIs

The optimized weights and thresholds obtained from Example 3 are used to evaluate the predictive capabilities of the method. With 5 days of SEM delta values, data for the 6th day (Day 0) is predicted using linear interpolation of the data from the 3 previous days. As shown in FIG. 7, the SEM delta values are weighted accordingly and are used to calculate the inequality.

### Example 5

### Detection and Prediction of DTIs

The method is able to detect DTIs with an accuracy of 79%, with a true positive rate of 90% and a false positive rate of 21%. The method is also able to predict the formation of DTIs, with an accuracy of 77%, with a true positive rate of 80% and a false positive rate of 23%.

While the invention has been described with reference to particular embodiments, it will be understood by those skilled in the art that various changes may be made and equivalents may be substituted for elements thereof without departing from the scope of the invention. In addition, many modifications may be made to a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

### Example 6

### Obtaining SEM Delta Values of Patients With and Without DTI at the Sacrum

A blinded clinical trial is conducted to measure the SEM delta values of patients with skin and tissue at increased risk of developing pressure injuries. SEM delta values are measured in patients at the sacrum over time, at a frequency of once a day. Patients are visually assessed daily for deep tissue injury (DTI). Patient data is categorized into two groups, patients who are diagnosed with DTI and patients who are yet to be diagnosed with DTI. For patients who are diagnosed with DTI, the SEM delta values data in the days leading up to diagnosis are aligned with the day of diagnosis being Day 0. Data from patients with suspected DTI (Table 1) are used to build a classification algorithm to detect and predict formation of DTIs, as shown in FIG. 2B.

TABLE 1
Exemplary SEM values measured at the center
of a wound at the sacrum in patients
where a DTI was discovered on Day 0.

| Study Day Relative | SEM Readings at center | |
|---|---|---|
| to Day DTI | of wound at sacrum | |
| discovered (Day 0) | Patient A | Patient B |
| Day −12 | 2.5 | — |
| Day −11 | 2.5 | — |
| Day −10 | 3.5 | — |
| Day −9 | — | — |
| Day −8 | 2.4 | — |
| Day −7 | 2.6 | — |
| Day −6 | 3.1 | 2.7 |
| Day −5 | — | 2.1 |
| Day −4 | 3.7 | 2.0 |
| Day −3 | 2.5 | 2.1 |
| Day −2 | 3.0 | 2.8 |
| Day −1 | 3.3 | 2.7 |
| Day 0 | 1.8 | 2.8 |
| Day 1 | 3.5 | 2.9 |
| Day 2 | 3.3 | 2.2 |
| Day 3 | 3.5 | — |
| Day 4 | 2.7 | — |

1. A method for detecting deep tissue injury (DTI) before it is visible on a patient's skin, comprising:
a) obtaining a set of sub-epidermal moisture (SEM) delta values at a location on the patient's skin at a predetermined frequency;
b) applying to each of the SEM delta values of the obtained set a predetermined weight;
c) calculating a first average SEM delta value of the N least-recent weighted SEM delta values;
d) calculating a second average SEM delta value of the M most-recent weighted SEM delta values;
e) comparing the difference between the first average and the second average SEM delta value with a predetermined threshold value; and
f) determining that there is DTI at the location on the patient's skin when the difference is greater than the predetermined threshold value.

2. (canceled)

3. The method of claim 1, wherein the predetermined frequency is once a day.

4.-6. (canceled)

7. The method of claim 1, wherein N is 4 and M is 2.

8. The method of claim 1, wherein the predetermined threshold value is a real number in the range of 0 to 1.

9. The method of claim 1, wherein the most recent SEM delta value is obtained by linear extrapolation of the K most-recent SEM delta values.

10.-12. (canceled)

13. A computer-implemented method for training a neural network for detection of deep tissue injury (DTI) before the injury is visible on a patient's skin, comprising:
a) for each patient in a first plurality of patients who have been diagnosed with a DTI, obtaining a first set of sub-epidermal moisture (SEM) delta values at a location on the patient's skin at a predetermined frequency before and up to the formation of the DTI;
b) for each patient in a second plurality of patients who have not been diagnosed with a DTI, obtaining a second set of SEM delta values at the same location on the patient's skin at the predetermined frequency;
c) applying a set of weights to each of the SEM delta values of the first and second set of obtained SEM delta values;
d) creating a training set comprising the first set of weighted SEM delta values and the second set of weighted SEM delta values of all patients in the first and second plurality of patients; and
e) training the neural network using the training set.

14. The method of claim 13, wherein the trained neural network outputs a set of optimized weights, comprising an optimized weight for each timepoint before the formation of a DTI, and wherein the optimized weights monotonically increase with time.

15. The method of claim 13, further comprising:
a) for each patient in the first and second plurality of patients,
i) calculating a first average SEM delta value of the N least-recent weighted SEM delta values in the set of SEM delta values;
ii) calculating a second average SEM delta value of the M most-recent weighted SEM delta values in the set of SEM delta values;
iii) calculating a difference value between the first average and the second average SEM delta value; and
iv) comparing the difference between the first average and the second average SEM delta value with a threshold value;
b) creating a training set comprising a first set of difference values in the first plurality of patients and a second set of difference values in the second plurality of patients; and
c) training the neural network using the training set.

16. The method of claim 13, wherein the predetermined frequency is once a day.

17. The method of claim 13, wherein the first and second set of SEM delta values comprises at least six SEM delta values each taken one day apart.

18. The method of claim 15, wherein N+M=6.

19. The method of claim 15, wherein N=4 and M=2.

20.-28. (canceled)

29. A computer-implemented method for predicting deep tissue injury (DTI) in a patient, the method comprising:
a) receiving, via an input device, a plurality of sub-epidermal moisture (SEM) delta values associated with the patient;
b) automatically inputting, via a processor, the plurality of SEM delta values into a trained model,
wherein the trained model is configured to calculate a probability value corresponding to the likelihood of the patient developing DTI; and
wherein the trained model is trained based on a set of training data comprising SEM delta data from a set of patients; and
c) outputting, via an output device, a prediction of the likelihood of the patient developing DTI based on the probability value.

30. The method of claim 29, wherein the trained model is trained by performing the steps comprising:
a) receiving a set of training data comprising:
1) a plurality of SEM delta values associated with a set of patients, wherein each patient in the set of patients has a known DTI status, and
2) a threshold value, wherein the threshold value is a number between 0 and 1;
b) automatically inputting the training data into an optimization algorithm to receive a plurality of optimal weight values; and
c) automatically updating the trained model with the plurality of optimal weight values.

31. The method of claim 30, wherein the optimization algorithm is configured to:
a) generate a plurality of ascending random numbers between 0 and 2 as a plurality of weight values;
b) input the training data and the plurality of weight values into the trained model to receive a set of predicted DTI statuses associated with the set of patients;
c) compare the predicted DTI statuses with the known DTI statuses associated with the set of patients;
d) calculate a true positive rate (TPR) and a false positive rate (FPR) based on the comparison, wherein the TPR is calculated as percentage of patients in the set of patients whose predicted DTI status matches their known DTI status, and the FPR is calculated as percentage of patients in the set of patients whose predicted DTI status does not match their known DTI status;
e) repeat steps a) to d) for a predetermined number of iterations to obtain a plurality of TPRs and FPRs;
f) identify an optimal TPR and FPR from the iterations; and
g) output the optimal plurality of weight values associated with the optimal TPR and FPR.

32. The method of claim 29, wherein the plurality of SEM delta values comprises SEM delta values from a predetermined number of days preceding the prediction.

33. The method of claim 31, wherein identifying the optimal TPR and FPR comprises minimizing the objective function of 1−TPR+FPR and satisfying the constraint of TPR»FPR.

34. (canceled)

35. (canceled)

36. A method for assessing risk of deep tissue injury (DTI) of a patient, the method comprising:
a) obtaining a plurality of sub-epidermal moisture (SEM) delta values associated with the patient;
b) inputting the plurality of SEM delta values into a trained model to receive a probability value, wherein the trained model is configured to calculate a probability value corresponding to the likelihood of the patient developing DTI, wherein the trained model is trained based on a set of training data comprising SEM delta values from a set of patients; and
c) assessing the risk of the patient developing DTI based on the probability value.

37.-51. (canceled)

52. A system for predicting or assessing deep tissue injury (DTI), comprising:
a) a Sub-Epidermal Moisture (SEM) scanner configured to make SEM measurements;
b) a processor electronically coupled to the SEM scanner and configured to receive the SEM measurements; and
c) a non-transitory computer readable media that is electronically coupled to the processor and comprises instructions stored thereon that, when executed on the processor, performs the steps of:
i) calculating a plurality of SEM delta values from the SEM measurements,
ii) automatically inputting, via a processor, the plurality of SEM delta values into a trained model to receive a probability value, wherein the trained model is configured to predict a probability value corresponding to a future occurrence of the patient developing DTI, and wherein the trained model is trained based on a set of training data comprising a plurality of SEM delta values associated with a set of patients;
iii) outputting, via an output device, a prediction of the future occurrence of the patient developing DTI based on the probability value.

53. The system of claim 52, wherein training the trained model comprises:
a) receiving, via an input device, the set of training data comprising:
i) a plurality of SEM delta values associated with a set of patients, and
ii) a threshold value, wherein each patient in the set of patients has a known DTI status, and wherein the threshold value is a number between 0 and 1;
b) automatically inputting, via a processor, the training data into an optimization algorithm to receive a plurality of optimal weight values; and
c) automatically updating, via a processor, the trained model with the plurality of optimal weight values.

54. The system of claim 53, wherein the optimization algorithm is configured to:
a) generate a plurality of ascending random numbers between 0 and 2 as a plurality of weight values;
b) input the training data and the plurality of weight values into the model to receive a set of predicted DTI statuses associated with the set of patients;
c) compare the predicted DTI statuses with the known DTI statuses associated with the set of patients;
d) calculate a true positive rate (TPR) and a false positive rate (FPR) based on the comparison, wherein the TPR is calculated as percentage of patients in the set of patients whose predicted DTI status matches their known DTI status, and the FPR is calculated as percentage of patients in the set of patients whose predicted DTI status does not match their known DTI status;
e) repeat steps a) to d) for a predetermined number of times as the number of iterations;
f) identify optimal TPR and FPR from all calculated TPRs and FPRs from the iterations; and
g) output the plurality of optimal weight values associated with the identified optimal TPR and FPR.

55. The system of claim 54, wherein the plurality of SEM delta values comprises SEM delta values from a predetermined number of days before the day of predicting PI.

56. The system of claim 54, wherein identifying optimal TPR and FPR comprises minimizing the objective function of 1−TPR+FPR and satisfying the constraint of TPR»FPR.

57. The system of claim 53, wherein the input device is the SEM scanner.

58.-70. (canceled)
